(19) United States
(12) Patent Application Publication
(10) Pub. No.: US 2014/0343517 A1
(43) Pub. Date: Nov. 20, 2014

(54) Negative Pressure Foot Bandage

(71) Applicant: Oakwell Distribution, Inc,, King of Prussia, PA (US)

(72) Inventor: Gayle A. Jameson, Roswell, GA (US)

(73) Assignee: Oakwell Distribution, Inc,, King of Prussia, PA (US)

(21) Appl. No.: 14/263,884

(22) Filed: Apr. 28, 2014

Related U.S. Application Data
(60) Provisional application No. 61/823,929, filed on May 16, 2013.

(51) Int. Cl. A61F 13/06

(52) U.S. Cl. CPC A61F 13/069
USPC 604/319

(57) ABSTRACT

A negative pressure foot bandage kit includes a non-woven gauze member, gel strips placed around the patient's foot to form a gasket proximal to the wound site, a polymer outer film member sealed around the foot and against the gel strips to encompass the wound site, and a vacuum port applied to the outer film member in register with a hole formed therein to apply a vacuum to the bandage. The gauze member is spoon-shaped and includes a handle portion that can be cut to size for enclosure within the bandage, with a mesh layer on both opposing sides. The gauze member provides a medium for the transport of fluids from the wound site engaged with the spoon portion through the handle portion engaged with the vacuum port. The gel strips are applied to the foot so that the outer film member can be sealed against the strips.

### CROSS-REFERENCE TO RELATED APPLICATIONS

This application claims domestic priority on U.S. Provisional Patent Application Ser. No. 61/823,929, filed on May 16, 2013, the content of which is incorporated herein by reference.

### FIELD OF THE INVENTION

The present invention relates generally to negative pressure bandages operable to remove exudates and fluids from a wound and, more particularly, to a bandage structure that is specifically configured for application to a human foot.

### BACKGROUND OF THE INVENTION

Negative pressure therapy has been utilized for the treatment of a variety of wounds by medical practitioners. Conventional negative pressure bandages are generally large in size and often require the use of complicated equipment such as suction pumps, vacuum pumps and complex electronic controllers to apply a negative pressure within the bandage to draw exudates and fluids away from the wound to a remote collection container. Typically, negative pressure therapy involves other associated equipment, such as the exudates/fluid collection canisters, liquid transporting conduits, and pressure regulators/transducers/sensors. As a result, negative pressure bandages and related equipment tends to be bulky and relatively costly. Such complexity typically requires professional placement of the bandage and connection to the pump and collection canister, followed by consistent, regular patient supervision and monitoring. Generally, negative pressure bandages are applied for approximately two days, at which time the bandage must be removed and replaced by professional technicians.

The rising costs of healthcare and of medical devices, such as negative pressure bandages, provide incentive to develop less expensive equipment, and procedures that are more easily utilized to reduce the costs associated with the use of negative pressure therapy while improving on the effectiveness of the therapy. Simplification of the procedures and the equipment can allow in-home use of such therapies with a minimum of professional supervision and monitoring of the patients. Furthermore, patients continue to demand devices that are more easily portable to allow travel and mobility while utilizing the therapy.

Conventional applications of negative pressure therapy to wound sites typically incurs the cutting of a porous foam pad to fit into the wound, followed by an application of an adhesive surgical drape over the pad and wound site to seal against the skin of the patient around the wound site. The fluids and exudates from the wound can be removed from the bandage to a remote location through an application of a vacuum to a connector fitted into the adhesive surgical drape, such as is shown in U.S. Pat. No. 5,636,643, granted on Jun. 10, 1997, in U.S. Pat. No. 5,645,081, granted on Jul. 8, 1997, and in U.S. Pat. No. 7,216,651, granted on May 15, 2007, all of which were issued to Louis Argenta. Alternatively, the foam pad can be utilized as a storage reservoir by incorporating a hydrophobic filter at the connector to prevent the fluids from leaving the bandage, as is reflected below in greater detail. Negative pressure therapy is provided commercially by at least KCI, Smith & Nephew, Kalypto, Medela, Mepilex and Convatec. An earlier negative pressure wound therapy embodiment is disclosed in U.S. Pat. No. 4,969,880, issued to David S. Zamierowski on Nov. 13, 1990.

The application of conventional negative pressure bandages to certain parts of the body presents substantial difficulties in applying the bandage to the wound site and also in maintaining a seal against the skin around the wound. Without the negative pressure bandage being sealed against the skin of the patient, the negative pressure system will not operate. Conventional practices with the negative pressure bandages, such as are described below, require the planar bandages to be cut, shaped and compromised with respect to the sealing portion of the bandage to fit against the contoured body part.

In U.S. Pat. No. 7,615,036, granted to Ashok Joshi, et al on Nov. 10, 2009, a negative pressure bandage is disclosed in which the bandage has a housing that is sealed to the body surface of the patient and defines a liquid retention chamber coupled to a vacuum source to apply a negative pressure on the liquid retention chamber so that the exudates and fluids are drawn into an absorptive material within the liquid retention chamber. This liquid retention chamber is located adjacent to the wound from which the exudates and fluids are removed.

Improvements to negative pressure wound therapy devices can be found in U.S. Patent Publication No. 2009/0299251 of John Buan published on Dec. 3, 2009, to enhance the sealing of the bandage to the body surface of the patient. In this negative pressure wound therapy device, a vacuum is applied to a collection chamber in which an absorptive pad is disposed to collect the exudates and fluids drawn away from the wound by the vacuum (negative pressure). To enhance the connection of the tubing extending between the vacuum pump and the negative pressure therapy device, an extended length connector is disclosed, which will accommodate connection when ace wrap or other coverings are applied to the exterior of the bandage.

In U.S. Pat. No. 7,361,184, granted on Apr. 22, 2008, to Ashok Joshi, an attempt to provide a self-contained negative pressure wound therapy device is provided so that the device does not require connection to a remote vacuum source. In this negative pressure wound dressing, an absorptive pad is also disposed in the fluid collection chamber, which is located adjacent to the wound, the negative pressure drawing the exudates and fluids away from the wound into the absorptive pad. Several early embodiments of negative pressure bandages can be found in U.S. Pat. No. 5,636,643, granted to Louis Argenta, et al on Jun. 10, 1997, all of which, however, utilize a single chamber configuration in which a vacuum is applied to the fluid collection chamber and the exudates and fluid is drawn away through tubing to a remote pump and fluid retention chamber.

In U.S. Patent Application Publication No. 2010/0268198 of John Buan, et al, published on Oct. 21, 2010, a negative pressure wound therapy device is disclosed for utilization with a wound on the human foot. In this '198 patent application, the foot bandage is applied in sequential steps with a wound interface layer applied to the surface of the wound and a non-woven absorption material placed above the wound against the wound interface layer. In situations where the wound is located at a position that the port is uncomfortable, a second piece of non-woven material can be added to the first piece of absorption material over the wound to allow for communication of the negative pressure from the wound site to the port. The housing material is then applied to the foot by placing the foot onto a first portion of the housing material and then wrapping the housing material around the toes to position the housing material so that the second portion of the housing material is folded back onto itself and the opposing portions of the gel gasket on the bandage is sealed together.

KCI Medical Products, Inc. markets a GranuFoam™ Bridge Dressing in which the dressing encompasses a bridge member that is sealed against the patient's leg with an elongated fluid collection chamber that terminates at a vacuum port that can be positioned on the patient's leg at a location remote from the wound site. The bridge member allows a connection of a source of negative pressure applied at the enlarged remote port to remove fluids from the wound site through communication with the elongated fluid collection chamber.

It would be desirable to provide a contoured negative pressure bandage that will be adapted to be affixed to a specific contoured body part to remove exudates and fluid from a wound located on that contoured body part.

### SUMMARY OF THE INVENTION

It is an object of this invention to overcome the aforementioned problems of the prior art in providing a negative pressure wound therapy apparatus that is specifically directed to treatment of a foot wound.

It is another object of this invention to provide a negative pressure wound therapy apparatus specifically applied to the human foot to remove fluids and exudates from a wound site anywhere on the foot.

It is an advantage of this invention that the negative pressure wound therapy for a foot wound can be provided as a kit with partially assembled components that can be applied to a foot wound by a professional care giver.

It is a feature of this invention that the negative pressure foot bandage kit includes a pre-assembled outer film member, a pre-assembled vacuum port, a gauze member, gel strips for sealing the outer film member against the human appendage, toe wedges for separating toes within the foot bandage, and tubing to connect the vacuum port to a remote vacuum pump.

It is another feature of this invention that the gauze member is formed with a generally circular spoon portion and an integral elongated handle portion projecting from the spoon portion.

It is another advantage of this invention that the gauze member can be utilized to transport fluids from a location on the underside of the human foot to a position on the upper side of the human foot where the vacuum port can be more effectively positioned.

It is still another feature of this invention that the gauze member is provided with a mesh liner on opposing sides of a non-woven gauze material.

It is still another advantage of this invention that the gauze member can be applied to a foot wound with either opposing side being in contact with the wound.

It is yet another feature of this invention that the handle portion of the gauze member can transport fluids from the spoon portion, which is in contact with the wound, to the vacuum port, which is in contact with the vacuum port.

It is yet another advantage of this invention that the vacuum port can be mounted on the outer film member at an appropriate location corresponding to a convenient position on the patient's foot for connection of the tubing from the remote vacuum pump.

It is still another feature of this invention that both the spoon portion and the handle portion of the gauze member can be cut to size for application to the specific wound site on the patient's foot such that the gauze member will be entirely contained within the outer film member encasing the patient's foot and connected to the vacuum pump.

It is yet another advantage of this invention that the application of the gauze member to a foot wound does not create a pinch point for the patient, particularly when the patient's wound is located on the underside of the patient's foot.

It is still another advantage of this invention that the application of the gauze member to a patient's foot wound facilitates the patient to walk while the negative pressure foot bandage is being worn by the patient.

It is still another object of this invention to provide a negative pressure foot bandage that includes an outer plastic film that wraps around the end of the foot to seal against itself and against gel strips placed around the foot above the wound site.

It is yet another object of this invention to provide a negative pressure foot bandage that utilizes a spoon-shaped gauze member that provides a medium for the transport of fluids from the wound site to the vacuum port at which negative pressure is applied to the foot bandage.

It is a further object of this invention to provide a method of applying a negative pressure foot bandage to treat a wound on a patient's foot.

It is yet another feature of this invention that the method of applying a negative pressure foot bandage includes the steps of applying gel strips around the patient's foot at a location that is proximal to the wound site, securing a non-woven gauze member to the wound site, placing an adhesive side of an outer film member on a bottom portion of the patient's foot beginning proximally of the gel strips, wrapping the outer film member around the patient's foot such that the adhesive side is sealed against itself and against the gel strips to define a sealed enclosure around the wound site, forming a hole in the outer film member in register with the remote end of the handle portion of the gauze member, attaching an adhesive side of a vacuum port to the outer film member, and drawing a vacuum on the vacuum port to extract fluids from the wound site through the gauze member to the vacuum port for removal of the fluids to a remote location.

It is a further feature of this invention that the outer film member is wrapped around the patient's foot to form a sealed enclosure encompassing the wound site and being sealed against gel strips placed proximal to the wound site.

It is still another object of this invention to provide a method of utilizing a negative pressure foot bandage providing negative pressure wound therapy to a wound site on a patient's foot that includes the steps of positioning a non-woven gauze member to said wound site where the gauze member has an enlarged spoon portion and an integral elongated handle portion extending away from the spoon portion. The gauze member is positionable in engagement with the wound site such that the distal end of the handle portion terminates at a location remote from said wound site. An adhesive side of an outer film member is placed on a bottom portion of said patient's foot and then wrapped around the patient's foot such that said adhesive side is sealed against itself to define a sealed enclosure around said wound site and covering the gauze member. A hole is formed in the outer film member in register with the distal of said handle portion of said gauze member so that a vacuum port can be mounted on the outer film member in communication with the hole formed in said outer film member for connection with a vacuum source to extract fluids from the wound site with said fluids being conveyed from said wound site along said handle portion to said vacuum port for removal to a remote location.

It is a further object of this invention to provide a negative pressure foot bandage that is sealable on a patient's foot to encompass a wound site to apply negative pressure wound therapy in a manner that is simple and effective in use.

These and other objects, features and advantages are accomplished according to the instant invention by providing a negative pressure foot bandage kit that includes a non-woven gauze member, gel strips placed around the patient's foot to form a gasket proximal to said wound site, a polymer outer film member sealed around the foot and against the gel strips to encompass the wound site, and a vacuum port applied to the outer film member in register with a hole formed therein over said gauze member to apply a vacuum to the bandage. The gauze member is spoon-shaped and includes a handle portion that can be cut to size for enclosure within the bandage, with a mesh layer on both opposing sides. The gauze member provides a medium for the transport of fluids from the wound site engaged with the spoon portion through the handle portion engaged with the vacuum port. The gel strips are applied to the foot so that the outer film member can be sealed against the strips.

### BRIEF DESCRIPTION OF THE DRAWINGS

The foregoing and other objects, features, and advantages of the invention will appear more fully hereinafter from a consideration of the detailed description that follows, in conjunction with the accompanying sheets of drawings. It is to be expressly understood, however, that the drawings are for illustrative purposes and are not to be construed as defining the limits of the invention.

FIG. 1 is an exploded view of the component parts of a negative pressure foot bandage incorporating the principles of the instant invention and being configured to extract fluids from a wound on a human foot;

FIG. 2 is a bottom plan view of the suction bell assembly having a two-part release paper covering the bottom surface of the planar skirt;

FIG. 3 is a side elevational view of the suction bell assembly shown in FIG. 2;

FIG. 4 is a top plan view of the suction bell assembly shown in FIG. 2, the tip of the stiffening layer being treated so as to be non-attached to the planar skirt to facilitate removal from the planar skirt;

FIG. 5 is a top plan view of the planar skirt forming part of the suction bell assembly;

FIG. 6 is a bottom plan view of the central bell member showing the planar rim;

FIG. 7 is a side elevational view of the central bell member shown in FIG. 6;

FIG. 8 is a top plan view of the central bell member;

FIG. 9 is a perspective view of the central bell member;

FIG. 10 is a plan view of the gauze member shown in FIG. 1;

FIG. 11 is a side elevational view of the gauze member shown in FIG. 10, the mesh liner on opposing sides of the gauze member being enlarged in thickness for purposes of clarity;

FIG. 12 is a perspective schematic view showing the bottom of a representative patient's foot having a wound thereon to depict the first step of the process of applying a negative pressure foot bandage incorporating the principles of the instant invention;

FIG. 13 is a perspective schematic view of the representative patient's foot shown in FIG. 12 to depict a subsequent step in the process of applying the negative pressure foot bandage;

FIG. 14 is a perspective schematic view of the top of a representative patient's foot as depicted in FIG. 13 to depict a subsequent step in the process of applying the negative pressure foot bandage;

FIG. 15 is a perspective schematic view of the representative patient's foot shown in FIG. 14 to depict a subsequent step in the process of applying the negative pressure foot bandage;

FIG. 16 is a perspective schematic view of the representative patient's foot depicting the conclusion of the application of the negative pressure foot bandage to a human foot according to the principles of the instant invention;

FIG. 17 is a perspective schematic view showing the bottom of a representative patient's foot having a wound thereon corresponding to two toes being amputated, the first step of the process of applying a negative pressure foot bandage incorporating the principles of the instant invention is depicted;

FIG. 18 is a perspective schematic view of the representative patient's foot shown in FIG. 17 to depict the subsequent step of applying the gauze member to the foot;

FIG. 19 is a perspective schematic view of the top of a representative patient's foot as depicted in FIG. 18 to depict the subsequent step of applying the outer film layer to the foot;

FIG. 20 is a perspective schematic view of the representative patient's foot shown in FIG. 19 to depict the subsequent step of encapsulating the negative pressure bandage on the foot; and

FIG. 21 is a perspective schematic view of the representative patient's foot depicting the conclusion of the application of the negative pressure foot bandage to a human foot according to the principles of the instant invention.

### DETAILED DESCRIPTION OF THE PREFERRED EMBODIMENT

Referring to the drawings, a negative pressure foot bandage incorporating the principles of the instant invention can best be seen. One of ordinary skill in the art will recognize that the scale of the components of the negative pressure bandages may be exaggerated to shown the details of the components. The negative pressure wound bandage 10 is specifically configured to be applied to the human foot to remove fluids and exudates from a wound site anywhere on the foot.

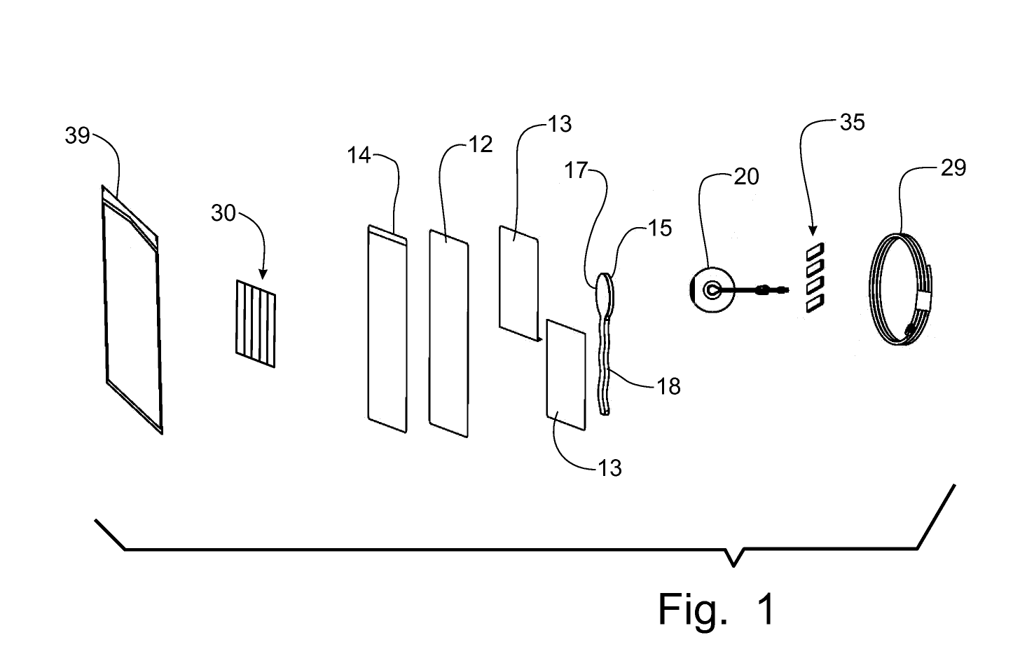

As depicted in the exploded view of FIG. 1, the component parts of the negative pressure foot bandage 10 include an outer film member 12 preferably formed of clear polyurethane film that is approximately 25 micrometers thick with an adhesive applied to one side of the film to establish an adhesive side and a non-adhesive side of the outer film member 12. A release liner 13 is applied to the adhesive side of the outer film member 12 to protect the adhesive side until application of the outer film member 12 is desired. The non-adhesive side of the outer film member 12 preferably has a stiffening layer 14 preferably formed of polyethylene terephthalate (PET) having a thickness of approximately 15 micrometers and adhered to the non-adhesive side by static attraction. The PET stiffening layer 14 provides sufficient stiffness for the outer film member 12 to enable handling of the outer film member 12 during the application steps, as defined below. After the outer film member 12 has been applied, the stiffening layer 14 can be removed from the outer film member 12 to enhance the comfort of the bandage 10 on the patient's foot.

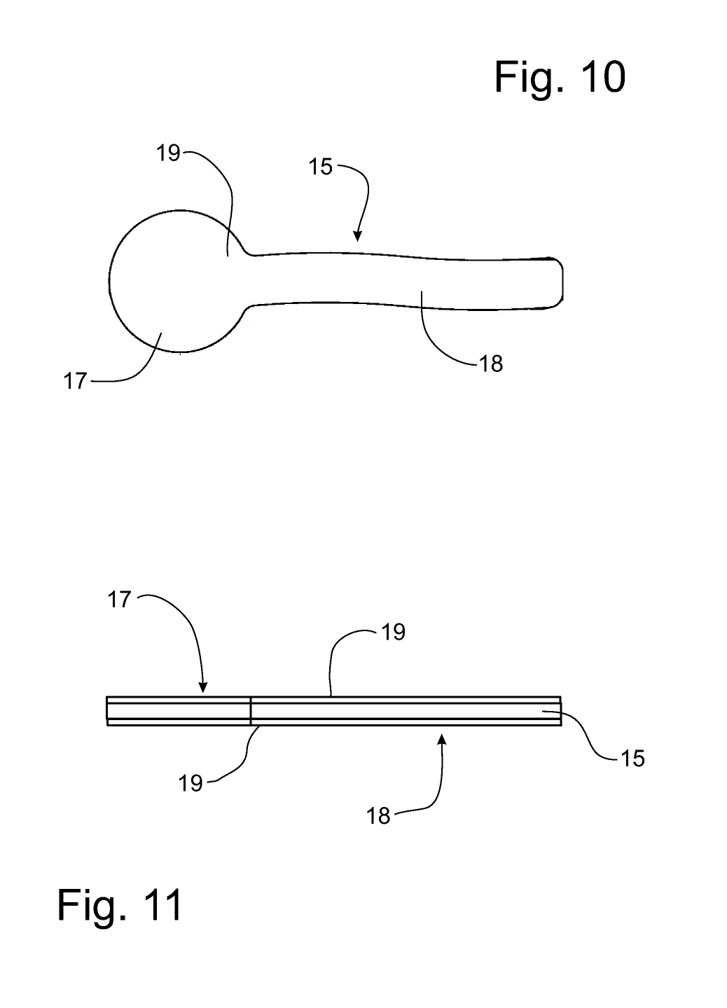

As depicted in FIGS. 1, 10 and 11, the negative pressure foot bandage 10 further includes a gauze member 15 formed from non-woven gauze material, which is used to interface with the wound site and permit the application of negative pressure to the wound site for the removal of fluids and exudates therefrom. Preferably the gauze member 15 is formed with a mesh liner 19 formed from non-woven polyurethane on at least one side of the gauze member 15 so that the mesh liner 19 can be positioned in contact with the wound site and protect the wound site from direct engagement with and integration into the non-woven gauze material. Preferably, the mesh liner 19 is applied to both sides of the gauze member 15 so that the gauze member 15 can be applied in either direction to the wound site.

The gauze member 15 is configured to have a generally circular, enlarged spoon portion 17, preferably having a diameter of approximately 100 millimeters, and an integral, elongated handle portion 18 projecting from the spoon portion 17 for approximately 220 millimeters. Preferably, the handle portion 18 will have a width of approximately 35 millimeters with the entire gauze member 15 being approximately 10 millimeters thick. The spoon portion 17 is applied to the wound site, particularly when the wound site is located on the bottom of the patient's foot, and the handle portion 18 is wrapped around the patient's foot to engage the vacuum port 20 for the application of negative pressure thereto. The mesh liner 19 permits the passage of fluids and exudates into the gauze material which moves along the gauze material through the handle portion 18 through the application of a vacuum applied through a vacuum port 20.

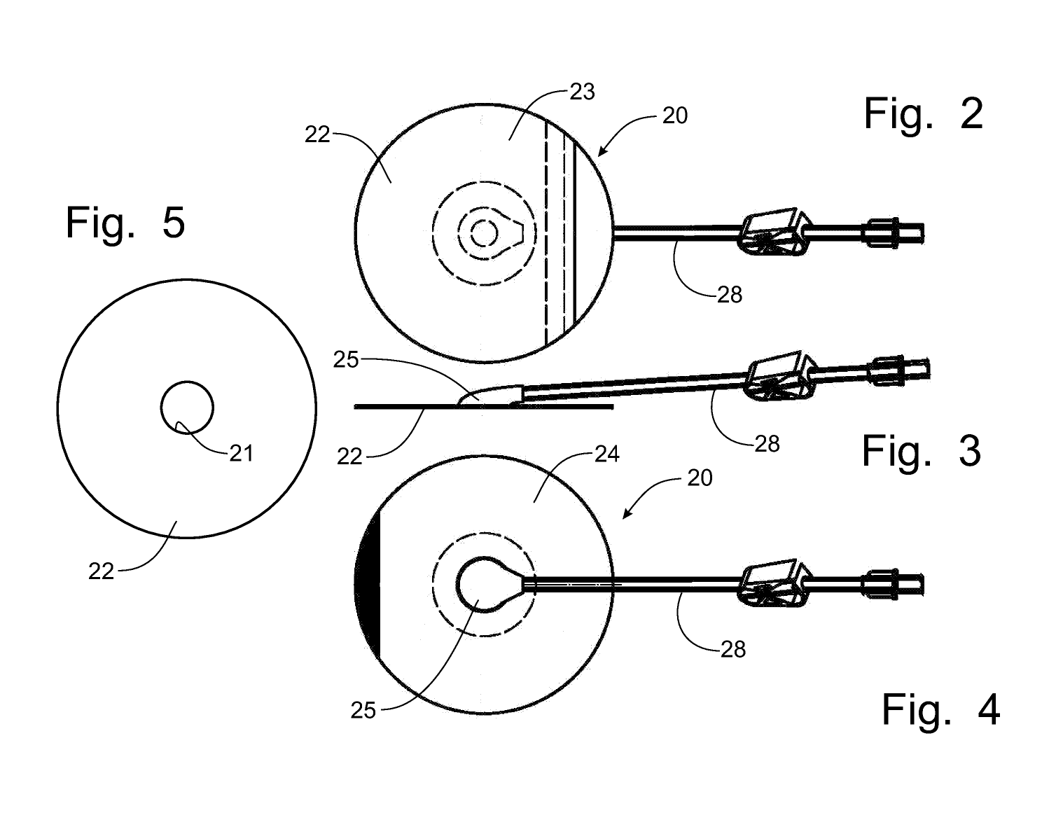

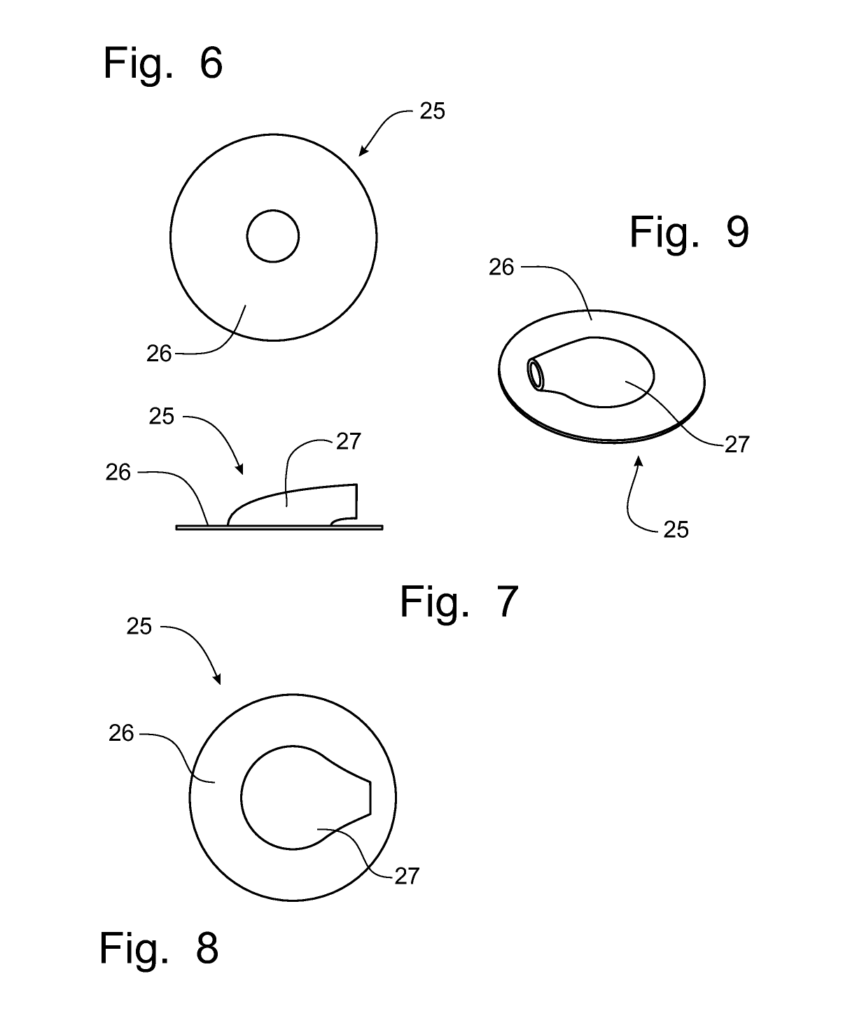

Referring now to FIGS. 1-5, the vacuum port, or suction bell assembly, 20 is applied to the non-adhesive side of the outer film member 12 after the outer film member 12 has been applied to the patient's foot and the stiffening layer 14 has been removed. The vacuum port 20 is preferably configured in a circular shape having a planar skirt 22 extending around a central bell member 25 that projects upwardly from a non-adhesive side of the port 20. The bell member 25 is preferably formed from polyvinyl chloride (PVC) having a thickness of approximately 0.7 millimeters to establish a stable, formed bell member 25 that will hold its shape when a vacuum is applied through the vacuum port. The central bell member 25 includes a planar rim 26 extending around the elevated suction bell 27 which carries a tube 28 connected thereto. The elevated suction bell 27 has a diameter of approximately 20 millimeters with the planar rim 26 extending around the suction bell 27 having a width of approximately 10 millimeters, providing a total diameter of the central bell member of approximately 40 millimeters.

The planar skirt 22 is formed from polyurethane in the same general manner as the outer film member 12. Thus, the planar skirt 22 has an adhesive side covered with a release liner 23 and has a stiffening layer 24 formed of PET statically attached to the outer non-adhesive side of the planar skirt 22. The planar skirt 22 preferably has a circular shape with a diameter of approximately 100 millimeters and a central opening 21 formed therein and having a diameter of approximately 21 millimeters. The central opening 21 is sized to receive the suction bell 27 such that when assembled the skirt 22 overlaps the planar rim 26 and is secured thereto with an adhesive.

The suction bell assembly 20 has the planar skirt 22 applied to the top surface of the planar rim 26 of the central bell member 25 with the elevated suction bell 27 projecting upwardly through the planar skirt 22 for attachment to a vacuum pump, not shown. The adhesive side of the planar skirt 22 outside of the planar rim 26 has an adhesive layer and is covered by the release layer 24. The stiffening layer 24 overlies the planar skirt 22 on the non-adhesive side and surrounds the elevated suction bell 27. When the vacuum port 20 is to be applied to the outer film member 12, as described in greater detail below, the release layer 24 is removed and the adhesive side of the planar skirt 22 is attached to the outer film member 12. After attachment, the stiffening layer 24 can be removed from the upper, non-adhesive side of the planar skirt 22 and removed over the elevated suction bell 27.

As best seen in FIG. 1, the negative pressure foot bandage 10 also includes a plurality of gel gasket strips 30 that can be applied in a manner described in greater detail below to establish a seal around the wound site with the outer film member 12. The negative pressure foot bandage 10 also includes a plurality of toe wedges 35 formed preferably from gauze material having an exterior surface of non-woven polyurethane mesh. The toe wedges are placed between the patient's toes to alleviate the collection of moisture between the patient's toes when the outer film member 12 is applied to the patient's foot.

The components of the negative pressure foot bandage 10 incorporating the principles of the instant invention are provided as a kit to be applied to the patient by a professional caregiver. The foot bandage kit 10 comes with the assembled outer film member 12, the assembled vacuum port 20, a gauze member 15, a plurality of gel strips 30, a plurality of toe wedges 35, and preferably extension tubing 29 to interconnect the suction bell tubing 28, all of which can be packed into an envelope 39 or other appropriate packaging and provided with a vacuum pump (not shown). The kit can also come with adhesive tape strips for the convenience of the professional caregiver when applying the negative pressure foot bandage 10 to a patient's foot.

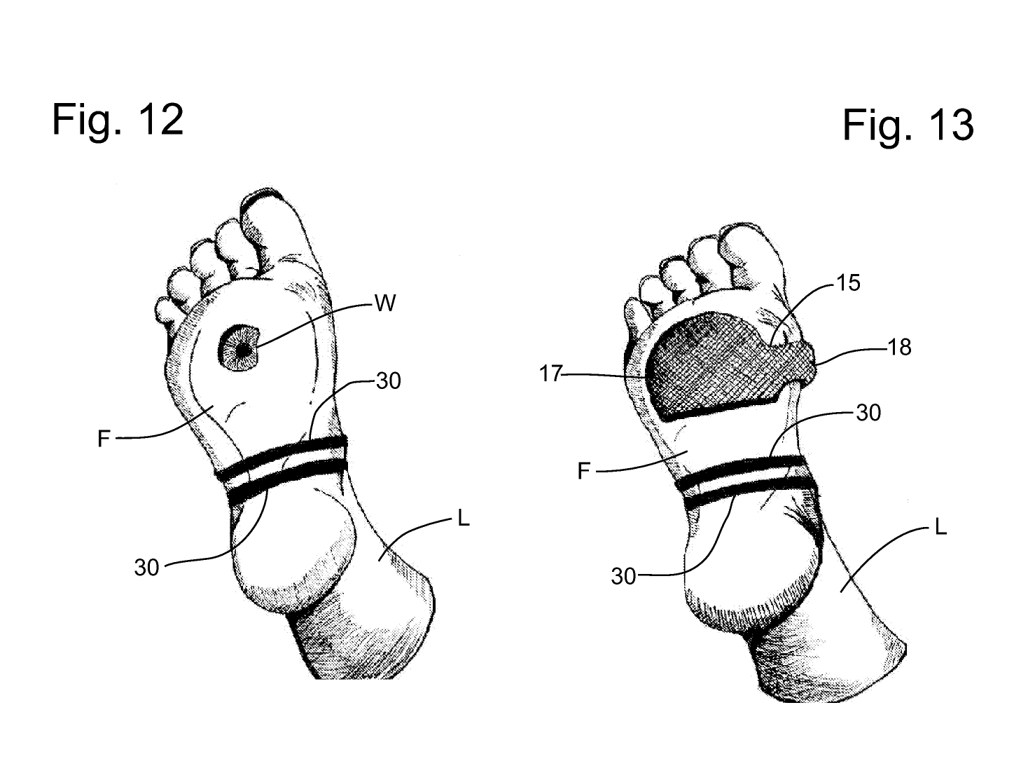

Referring now to the perspective schematic views of a representative patient's foot in FIGS. 12-16, the process of applying the negative pressure foot bandage 10 to a wound W on the patient's foot F can best be seen. The professional caregiver first applies the gel strips 30, as depicted in FIG. 12, onto the patient's foot F in a manner that surrounds the patient's foot at a location that is proximal to the wound site W. In other words, the gel strips 30 are applied to the patient's foot F to provide a gasket that will be located between the patient's leg L and the wound site W, but in a location that will be engaged by the outer film member 12 when the outer film member 12 is applied to the patient's foot, as described in greater detail below.

As shown in FIG. 13, the professional caregiver then applies the gauze member 15 in an orientation that applies the spoon portion 17 to the wound site. If the wound is a toe amputation, the spoon portion 17 can be applied to the bottom of the patient's foot F and the handle portion 18 positioned to overlap the wound site. The professional caregiver can utilize the adhesive tape strips (not shown) to secure the gauze member 15 in the selected orientation on the patient's foot F. For wound sites that are on the bottom portion of the patient's foot, as depicted in FIGS. 12-16, the handle portion 18 can be wrapped around the side of the patient's foot to wrap around and be located along the top surface of the patient's foot. For wound sites that are on the top portion of the patient's foot, the spoon portion 17 can be positioned over the wound site and the handle 18 oriented to the position where the vacuum port 20 is to be located. Whatever the orientation or location of the gauze member 15, the handle portion 18 can be cut to size so that the gauze member 15 extends from the wound site to the vacuum port 20. The use of the gauze member 15 does not create a pinch point for the patient, particularly with respect to a wound on the bottom of the patient's foot, thus facilitating walking while the bandage is worn.

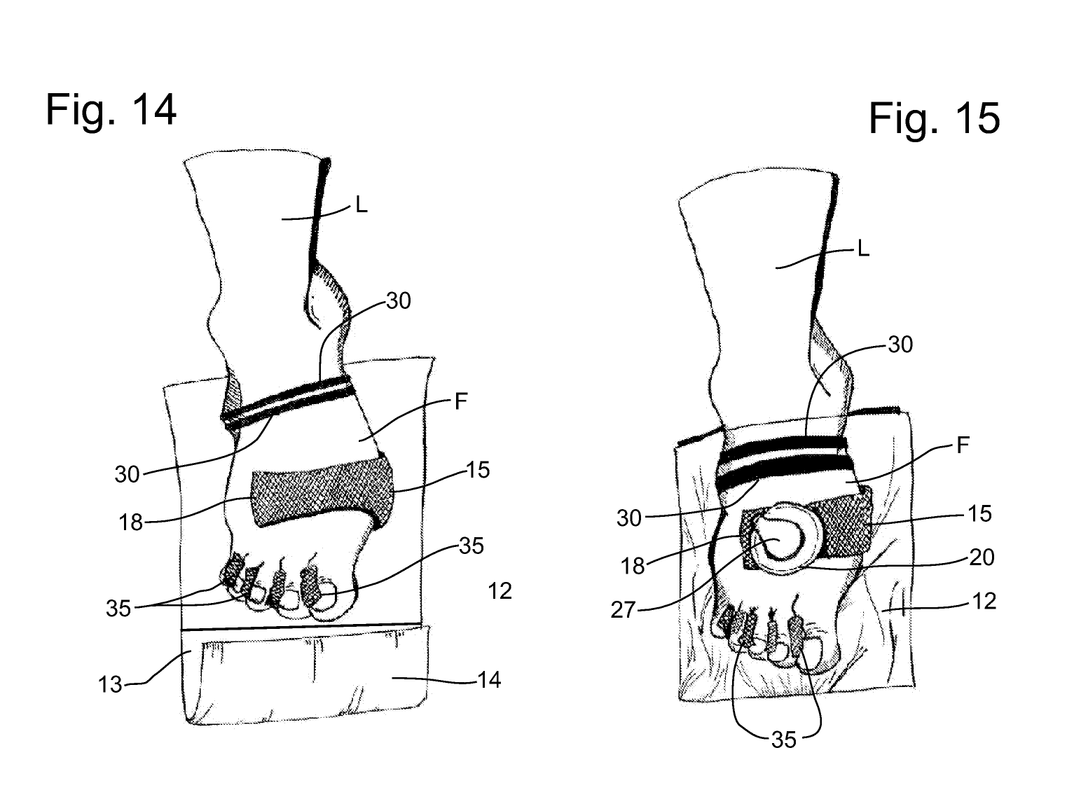

As depicted in FIG. 14, the toe wedges 35 are then inserted between the patient's toes to prevent a build-up of moisture between the patient's toes while the negative pressure foot bandage 10 is applied to the patient's foot. The outer film member 12 can then be applied to the patient's foot. A portion of the release liner 13 can be removed from the adhesive side of the outer film member 12 and the outer film member 12 applied to the bottom of the patient's foot F. Since most wound sites are found on the bottom of the patient's foot, the first portion of the outer film member 12 applied to the bottom of the patient's foot will typically cover the spoon portion 17 of the gauze member 15 and the wound site. This first portion of the outer film member 12 must overlap the gel strips 30 placed on the bottom portion of the patient's foot such that the gel strips 30 are sealed between the outer film member 12 and the patient's foot.

The outer film member 12 is then extended past the patient's toes and then folded over top of the patient's foot F, leaving a short distance between the ends of the patient's toes and the fold line of the outer film member 12, as is reflected in FIG. 15. The remainder of the release liner 13 is then removed from the outer film member 12 and the exposed adhesive side engaged against the top surface of the patient's foot, extending past the location of the gel strips 30 on the top surface of the patient's foot to form a seal between the top portion of the patient's foot and the outer film member 12. The outer film member 12 is then sealed against itself along the exterior sides of the patient's foot and against the gel strips 30 along the sides of the patient's foot. The end result is that the outer film member forms a sealed wrapping around the patient's foot from the toes toward the patient's leg, but extending past the gel strips to form a seal around the patient's foot. The stiffening layer 14 can then be removed from the non-adhesive side of the outer film member 12 to reduce the overall thickness of the outer film member 12 forming an enclosure around a portion of the patient's foot.

The professional caregiver will then cut a small hole about 25 millimeters in diameter into the outer film member 12 over top of the captured gauze member 15, most typically over the handle portion 18 thereof. The vacuum port 20 is then applied to the outer film member 12 with the elevated suction bell 27 aligned over the small hole cut into the outer film member 12. The release liner 23 is removed from the vacuum port 20 and the skirt 22 applied to the non-adhesive side of the outer member 12 with the elevated suction bell 27 registered with the hole cut into the outer film member 12, as is shown in FIG. 15. The stiffening layer 24 on the exterior surface of the vacuum port 20 can then be removed. The tubing 28 on the vacuum port 20 can subsequently be connected to the remote vacuum pump (not shown) using, if necessary, the tubing 29 provided in the kit to make the connection.

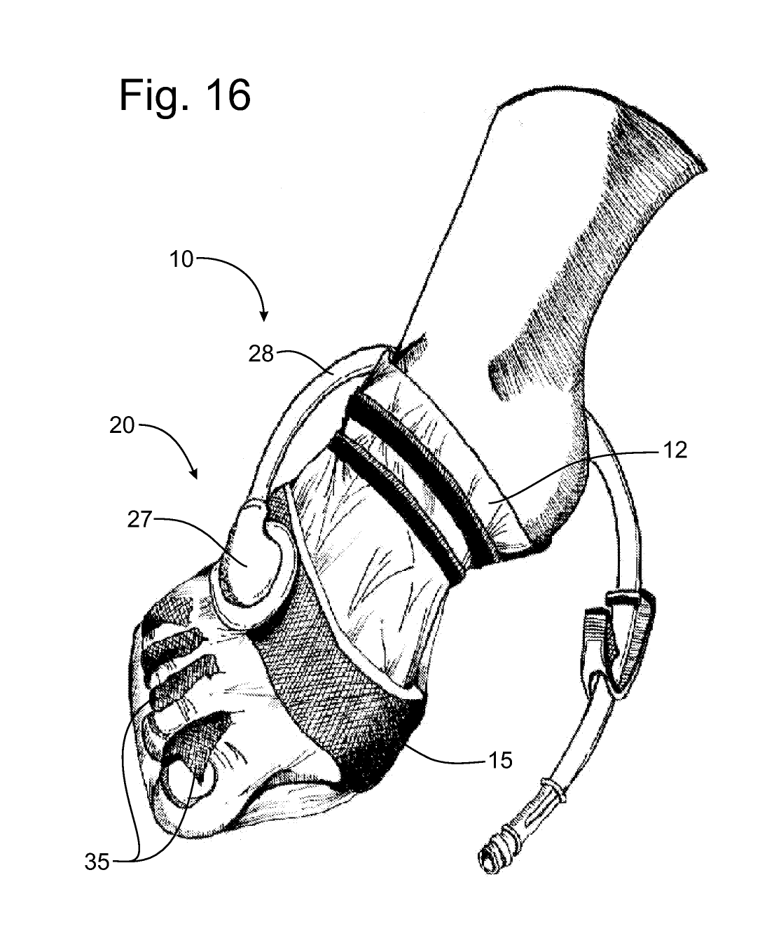

As is reflected in FIG. 16, the outer film member 12 can be tucked around the foot F to minimize the extraneous edges of the negative pressure foot bandage 10 that can be caught on an object and disrupt the seal or tear the outer film cover 12. The application of a vacuum by the pump (not shown) to the elevated suction bell 27 places the entire enclosure, defined by the outer film member 12 sealed against itself and against the gel strips, under negative pressure. The fluids and exudates are drawn from the wound site through the non-woven polyurethane mesh liner 19 into the gauze material and then through the gauze member 15 to the vacuum port and then through the elevated suction bell 27 and attached tubing 28 to a canister (not shown) associated with the vacuum pump (not shown) for removal from the wound site.

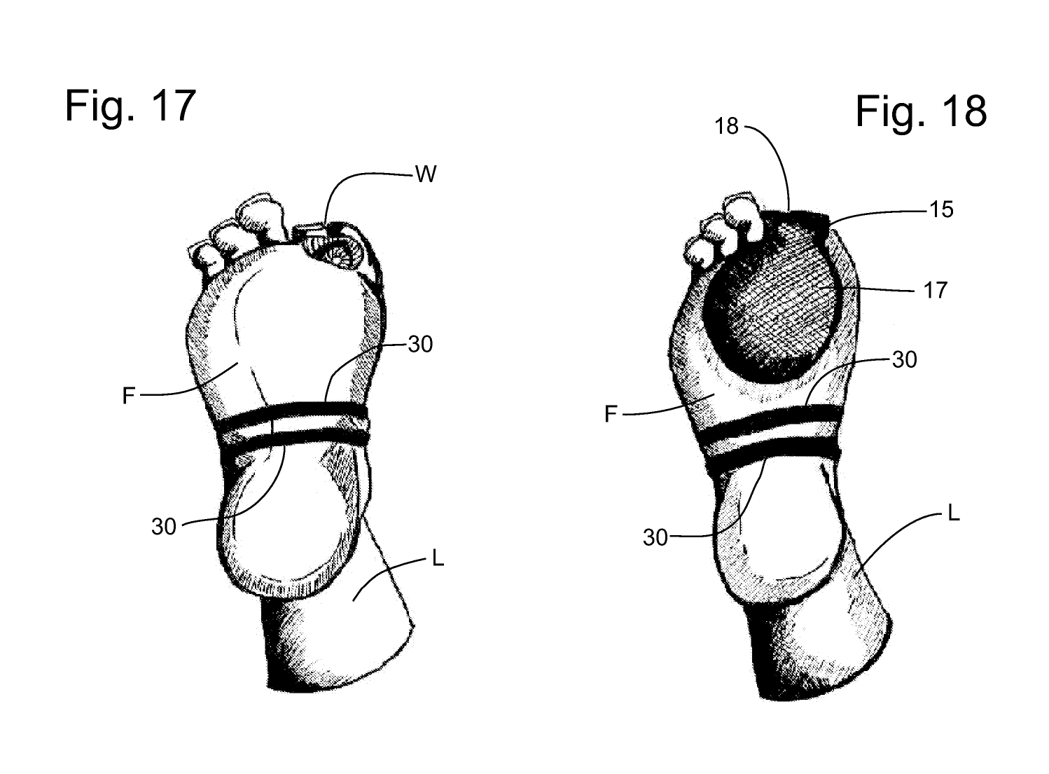

The negative pressure foot bandage 10 can also be utilized to treat toe amputations as is reflected in FIGS. 17-21. Similarly to the description above with respect to the application of the negative pressure foot bandage 10 for a wound on the bottom of the foot F as shown in FIGS. 12-16, the process can begin with the application of gel strips 30 around the circumference of the foot F at some location between the wound W and the leg L so that the gel strips 30 can form a seal between the foot F and the outer film member 12 when the application of the negative pressure foot bandage 10 is completed.

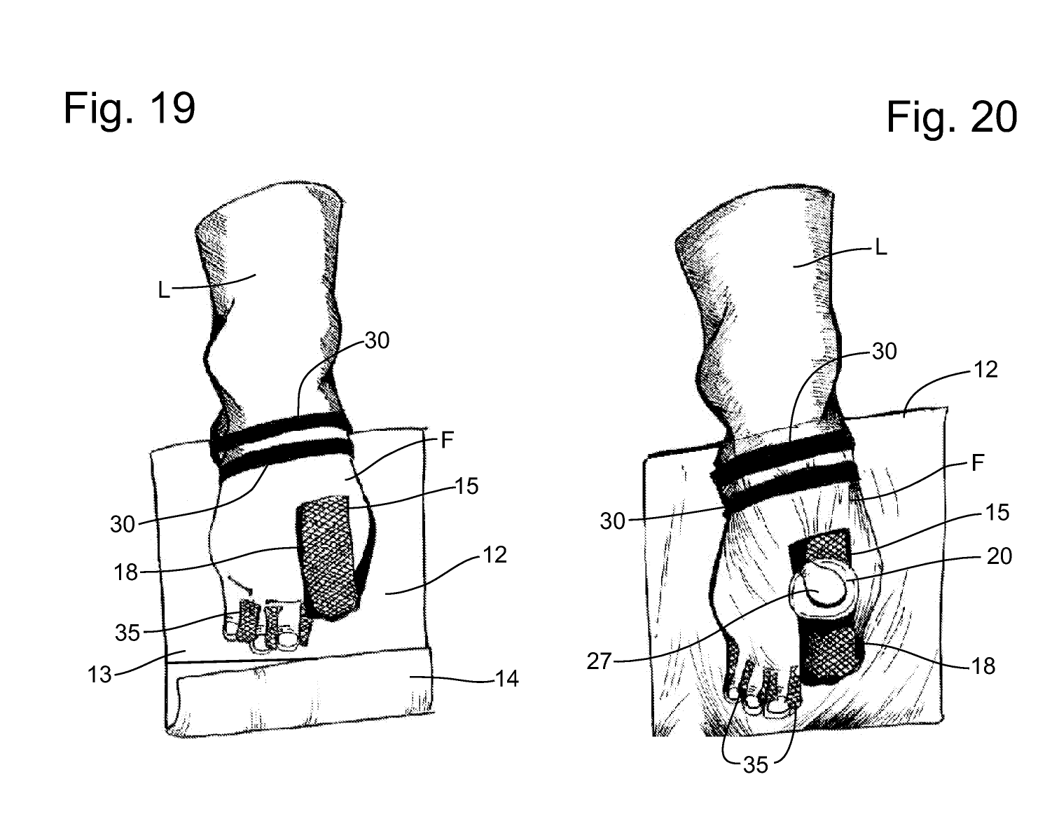

As reflected in FIGS. 18 and 19, the gauze member 15 is positioned such that the spoon portion 17 is located on the bottom of the foot F and the handle portion 18 is wrapped over the amputation wound site W to extend over the top of the foot F. Preferably, the gauze member 15 will be secured with adhesive strips (not shown) to maintain the positioning of the spoon and handle portions 17, 18 of the gauze member 15 during the subsequent steps in applying the negative pressure foot bandage 10 to the patient's foot F.

As is shown in FIGS. 19 and 20, the outer film member 12 is applied in substantially the same manner as described above with respect to the application of the bandage 10 to treat a wound on the bottom of the foot F as depicted in FIGS. 12-16. First, toe wedges 35 are preferably inserted between the patient's toes to prevent a build-up of moisture between the patient's toes while the negative pressure foot bandage 10 is applied to the patient's foot. The outer film member 12 can then be applied to the patient's foot F by removing a portion of the release liner 13 from the adhesive side of the outer film member 12 and applying the adhesive side of the outer film member 12 to the bottom of the patient's foot F. This first portion of the outer film member 12 on the bottom of the foot F must overlap the gel strips 30 placed on the bottom portion of the patient's foot so that the gel strips 30 will be sealed between the outer film member 12 and the patient's foot F.

The outer film member 12 is then folded over the patient's toes, leaving a short distance between the ends of the patient's toes and the fold line of the outer film member 12, as is reflected in FIG. 20, and the remaining portion of the adhesive side of the outer film member 12 is pressed onto the top surface of the patient's foot F, extending past the location of the gel strips 30 on the top surface of the patient's foot to form a seal between the top portion of the patient's foot F and the outer film member 12. The outer film member 12 is then sealed against itself along the exterior sides of the patient's foot and against the gel strips 30 along the sides of the patient's foot to form a sealed wrapping around the patient's foot from the toes toward the patient's leg, but extending past the gel strips to form a seal around the patient's foot F. The stiffening layer 14 can then be removed from the non-adhesive side of the outer film member 12 to reduce the overall thickness of the outer film member 12 forming an enclosure around a portion of the patient's foot.

The professional caregiver will then cut a small hole about 25 millimeters in diameter into the outer film member 12 over top of the captured handle portion 18 of the gauze member 15. The vacuum port 20 is then applied to the outer film member 12 with the elevated suction bell 27 aligned over the small hole cut into the outer film member 12. The release liner 23 is removed from the vacuum port 20 and the skirt 22 applied to the non-adhesive side of the outer member 12 with the elevated suction bell 27 registered with the hole cut into the outer film member 12, as is shown in FIG. 20. The stiffening layer 24 on the exterior surface of the vacuum port 20 can then be removed. The tubing 28 on the vacuum port 20 can be connected to the remote vacuum pump (not shown).

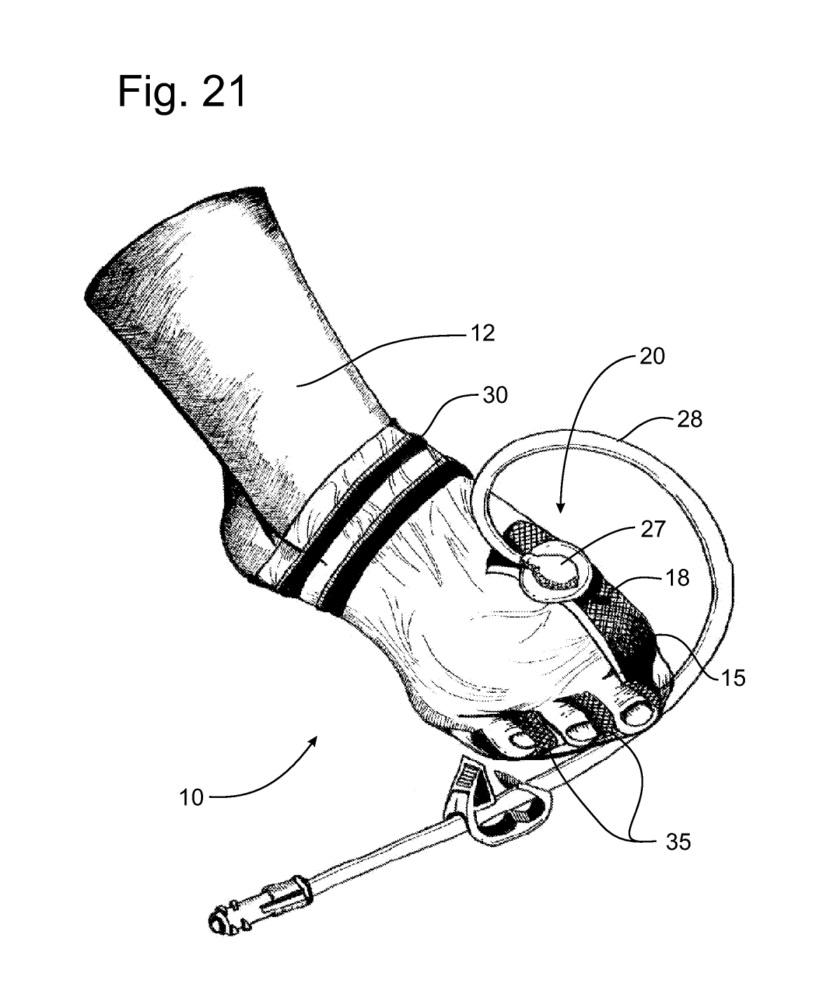

As is depicted in FIG. 21, the outer film member 12 can be tucked around the foot F to minimize the extraneous edges of the negative pressure foot bandage 10 that can be caught on an object and disrupt the seal or tear the outer film cover 12. The application of a vacuum by the pump (not shown) to the elevated suction bell 27 places the entire enclosure, defined by the outer film member 12 sealed against itself and against the gel strips, under negative pressure. The fluids and exudates are drawn from the wound site W through the non-woven polyurethane mesh liner 19 into the gauze material and then through the gauze member 15 to the vacuum port 20 and then through the elevated suction bell 27 and attached tubing 28 to a canister (not shown) associated with the vacuum pump (not shown) for removal from the wound site W.

It will be understood that changes in the details, materials, steps and arrangements of parts which have been described and illustrated to explain the nature of the invention will occur to and may be made by those skilled in the art upon a reading of this disclosure within the principles and scope of the invention. The foregoing description illustrates the preferred embodiments of the invention; however, concepts, as based upon the description, may be employed in other embodiments without departing from the scope of the invention. The invention is not otherwise limited, except for the recitation of the claims set forth below.

Having thus described the invention, what is claimed is:

1. A negative pressure foot bandage for applying negative pressure wound therapy to a wound site on a patient's foot, comprising:
a non-woven gauze member having a spoon portion and an integral handle portion extending from said spoon portion, said gauze member including a mesh layer on at least one side of said gauze member, said gauze member being positionable with said spoon portion being applied to said wound site and said handle portion extending away from said spoon portion;
gel strips placed around the patient's foot to form a gasket proximal to said wound site;
a polymer outer film member having an adhesive side and a non-adhesive side, said adhesive side being applied to said patient's foot in a manner that wraps around the patient's foot to form a sealed enclosure encompassing said wound site and being sealed against said gel strips; and
a vacuum port applied to said outer film member in register with a hole formed in said outer film member over said gauze member, said vacuum port having a lower adhesive side secured against said outer film member around said hole to permit an application of negative pressure to said vacuum port to extract fluids from said wound site through said gauze member.

2. The negative pressure foot bandage of claim 1 wherein said gauze member, said gel strips, said outer film member and said vacuum port are provided as a kit.

3. The negative pressure foot bandage of claim 1 wherein said vacuum port comprises:
a planar skirt having an adhesive side and a non-adhesive side, said planar skirt including a central opening formed therein;
a central bell member including an elevated suction bell and a planar rim portion extending around said elevated suction bell, said planar skirt being attached to said planar rim with said elevated suction bell projecting through said opening in said planar skirt; and
a release liner mounted on said adhesive side of said planar skirt to be removed when said vacuum port is to be applied to said outer film member.

4. The negative pressure foot bandage of claim 1 wherein said handle portion of said gauze member extends from the spoon portion to the location of the vacuum port to provide a medium for the transport of fluids from said wound site to the vacuum port.

5. The negative pressure foot bandage of claim 4 wherein said handle portion can be cut to length to accommodate the location of both the wound site and the position of the vacuum port.

6. The negative pressure foot bandage of claim 1 wherein said outer film member is applied to the patient's foot by wrapping the outer film strip around the patient's toes.

7. The negative pressure foot bandage of claim 6 wherein the outer film member is applied with the adhesive side pressed against the underside of the patient's foot, then wrapped around the patient's toes and applied against the upper side of the patient's foot.

8. The negative pressure foot bandage of claim 7 wherein the outer film member seals against itself and against the gel strips positioned around the patient's foot.

9. The negative pressure foot bandage of claim 8 wherein said negative pressure foot bandage is provided in a kit including said outer film member, said vacuum port, said gauze member, and said gel strips for sealing the outer film member against the human appendage.

10. The negative pressure foot bandage of claim 9 further comprising toe wedges for separating toes within the foot bandage.

11. A method of applying a negative pressure foot bandage to a patient's foot to provide negative pressure wound therapy to a wound site on said patient's foot, comprising the steps of:
applying gel strips around said patient's foot at a location that is proximal to said wound site;
securing a non-woven gauze member to said wound site, said gauze member having an enlarged spoon portion positioned in engagement with said wound site and an integral elongated handle portion extending away from said spoon portion and terminating at a location remote from said wound site;
placing an adhesive side of an outer film member on a bottom portion of said patient's foot beginning proximally of said gel strips;
wrapping said outer film member around said patient's foot such that said adhesive side is sealed against itself and against said gel strips to define a sealed enclosure around said wound site and around said gauze member;
forming a hole in said outer film member in register with the remote end of said handle portion of said gauze member;
attaching an adhesive side of a vacuum port to said outer film member, said vacuum port including a planar skirt positioned around said hole in said outer film member to seal said vacuum port to said outer film member and a central bell member including an elevated suction bell in register with said hole formed in said outer film member; and
drawing a vacuum on said vacuum port to extract fluids from said wound site through said gauze member to said vacuum port for removal of said fluids to a remote location.

12. The method of claim 11 wherein said securing step includes the step of:
positioning a mesh liner on said gauze member against said wound site, said mesh liner permitting the passage of fluids from said wound site into said gauze member for travel to said vacuum port during said drawing step.

13. The method of claim 12 wherein said securing step further includes the step of:
trimming said handle portion to a suitable length to terminate at a predetermined remote location.

14. The method of claim 13 wherein said handle portion of said gauze member provides a medium for the transport of fluids from said wound site to the vacuum port.

15. The method of claim 14 further comprising the step of:
placing toe wedges between the patient's toes prior to said placing step.

16. The method of claim 14 wherein said forming step is accomplished by selecting a site on said outer film member that will accommodate allowing the patient to walk while the negative pressure foot bandage is applied to the patient's foot.

17. A method of utilizing a negative pressure foot bandage providing negative pressure wound therapy to a wound site on a patient's foot, comprising the steps of:
positioning a non-woven gauze member to said wound site, said gauze member having an enlarged spoon portion and an integral elongated handle portion extending away from said spoon portion, said gauze member being positioned in engagement with said wound site with a distal end of said handle portion terminating at a location remote from said wound site;
placing an adhesive side of an outer film member on a bottom portion of said patient's foot;
wrapping said outer film member around said patient's foot such that said adhesive side is sealed against itself to define a sealed enclosure around said wound site and around said gauze member;
forming a hole in said outer film member in register with the distal of said handle portion of said gauze member;
attaching a vacuum port to said outer film member in register with said hole formed in said outer film member; and
drawing a vacuum on said vacuum port to extract fluids from said wound site with said fluids being conveyed from said wound site along said handle portion to said vacuum port for removal to a remote location.

18. The method of claim 17 wherein said positioning step locates said spoon portion of said gauze member in engagement with said wound site such that said fluids are conveyed along said handle portion to the distal end thereof.

19. The method of claim 18 further comprising the step of:
applying gel strips around said patient's foot at a location that is proximal to said wound site, said placing step beginning proximally of said gel strips and said wrapping step sealing said adhesive side of said outer film member against itself and against said gel strips.

20. The method of claim 19 wherein said forming step is accomplished by selecting a site on said outer film member corresponding to an upper portion of said patient's foot so that the patient may walk while the negative pressure foot bandage is applied to the patient's foot.
